(19) United States
(12) Patent Application Publication
(10) Pub. No.: US 2006/0229701 A1
(43) Pub. Date: Oct. 12, 2006

(54) Apparatus for use with expandable stents

(75) Inventors: Hitinder S. Gurm, Ann Arbor, MI (US); Jay Yadav, Hunting Valley, OH (US); Ji-Feng Chen, Lakewood, OH (US)

Correspondence Address: TAROLLI, SUNDHEIM, COVELL & TUMMINO L.L.P., 1300 EAST NINTH STREET, SUITE 1700, CLEVEVLAND, OH, 44114, US

(73) Assignee: The Cleveland Clinic Foundation

(21) Appl. No.: 11/372,932

(22) Filed: Mar. 10, 2006

Related U.S. Application Data
(60) Provisional application No. 60/661,424, filed on Mar. 14, 2005.

(51) Int. Cl. A61F 2/06

(52) U.S. Cl. 623/1.11

(57) ABSTRACT

An apparatus for use with an expandable stent having oppositely disposed first and second terminal edges. The apparatus has a catheter shaft with a lumen, and an inflatable balloon with oppositely disposed first and second end portions and an internal chamber. The internal chamber is in fluid communication with the lumen of the catheter shaft, and receives inflation fluid to inflate the balloon. The balloon has cylindrical inner and outer surfaces that extend between the end portions. The cylindrical inner surface is secured to the catheter shaft at first and second attachment sites adjacent the first and second end portions, respectively. At least one of the first and second end portions of the balloon has a toroidal shape defined by an arcuate end surface that intersects the cylindrical inner and outer surfaces at a common radial plane. A respective one of the attachment sites is located adjacent the one end portion lying adjacent the common radial plane. The balloon is positionable within the stent so that one of the terminal edges of the stent lies in the common radial plane to ensure that, when the balloon is expanded, the one terminal edge receives full and uniform expansion pressure.

### RELATED APPLICATION

This application claims priority from U.S. Provisional Patent Application Ser. No. 60/661,424, filed Mar. 14, 2005, the subject matter of which is incorporated herein by reference.

### TECHNICAL FIELD

The present invention relates to an apparatus for use with medical stents, and is particularly directed to an angioplasty balloon for use with expandable stents.

### BACKGROUND OF THE INVENTION

Balloon catheters are commonly used to treat certain conditions of a blood vessel, such as partial or total occlusion or lesion of the vessel which may be caused by, for example, atherosclerotic plaques or thrombosis. In an angioplasty procedure, the balloon portion of the catheter is advanced over a guidewire to the site of the occlusion and is inflated to compress the occlusion and thereby restore normal blood flow through the vessel. In some instances, an expandable stent may be implanted in the blood vessel to prevent the occlusion from recurring. A balloon catheter is commonly used to deliver and deploy the stent. The stent is typically mounted in its unexpanded state on the balloon portion of the catheter, delivered to the site of the occlusion, and then deployed or implanted in the vessel by inflating the balloon portion to stretch the stent into an expanded state.

Currently employed angioplasty balloon systems have a common profile. A cylindrical balloon is typically attached to a catheter shaft at a distal end, and includes a tapered edge having a variable diameter that leads from the catheter shaft to a balloon shoulder spaced radially apart from the catheter shaft. The balloon shoulder is the length of balloon that extends beyond the edge of the stent and the main body of the balloon. This balloon profile is necessitated by the need to maintain a strong bond between the balloon and the catheter shaft. Attempts to modify current angioplasty balloon profiles have been limited by safety concerns. For instance, attempts to shorten the tapered edge have been tempered by concerns of inadequate stent edge apposition - a phenomenon that can predispose the vessel to stent thrombosis.

Similarly, the safety of current angioplasty balloon profiles is limited by the occurrence of edge restenosis. Edge restenosis is believed to occur in response to pressure applied to the vessel wall by an angioplasty balloon shoulder overhanging beyond the stent edge and mushrooming out beyond the stent edge to contact the vessel wall. Such injury is unavoidable with current balloon systems, given the need for high-pressure balloon inflation to achieve adequate stent deployment.

### SUMMARY OF THE INVENTION

The present invention relates to an apparatus for use with expandable stents. More particularly, the present invention is directed to an angioplasty balloon for use with an expandable stent having oppositely disposed first and second terminal edges. The apparatus comprises a catheter shaft having a lumen, and an inflatable balloon having oppositely disposed first and second end portions and an internal chamber. The internal chamber is in fluid communication with the lumen in the catheter shaft, and receives inflation fluid to inflate the balloon. The balloon, includes cylindrical inner and outer surfaces that extend between the end portions. The cylindrical inner surface of the balloon is secured to the catheter shaft at first and second attachment sites adjacent the first and second end portions, respectively. At least one of the first and second end portions of the balloon has a toroidal shape defined by an arcuate end surface that intersects the cylindrical inner and outer surfaces at a common radial plane. A respective one of the attachment sites is located adjacent the one end portion lying adjacent to the common radial plane. The balloon is positionable within the stent so that one of the terminal edges of the stent lies in the common radial plane to ensure that, when the balloon is expanded, the one terminal edge receives full and uniform expansion pressure.

In one aspect of the present invention, the second end portion of the balloon has a toroidal shape defined by an arcuate end surface that intersects the cylindrical inner and outer surfaces at a second common radial plane. A respective one of the attachment sites is located adjacent the second end portion lying adjacent the second common radial plane. The balloon is positionable within the stent so that the other of the terminal edges of the stent lies in the second radial plane to ensure that, when the balloon is expanded, the other terminal edge receives full and uniform expansion pressure.

In another aspect of the present invention, the apparatus is used in a bodily vessel.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 is a cross-sectional view of an apparatus constructed in accordance with the present invention, with the apparatus being shown in an inflated configuration;

FIG. 2 is a cross-sectional view of an alternative embodiment of the apparatus, with the apparatus being shown in an inflated configuration; and

FIG. 3 is a cross-sectional view of another alternative embodiment of the apparatus, with the apparatus being shown in an inflated configuration.

### DETAILED DESCRIPTION

The present invention relates to an apparatus 10 for use with expandable stents. More particularly, the present invention is directed to an angioplasty balloon for use with an expandable stent 12 having oppositely disposed first and second terminal edges 14 and 16. As described more fully below, the apparatus 10 includes a balloon catheter that is particularly useful in delivering and deploying a stent in a bodily vessel. The invention may be used with any conventional balloon catheter delivery system, including an over-the-wire balloon system or a rapid exchange balloon system. In addition, the invention may be used to deploy any conventional balloon-deployable stent. Therefore, the scope of the present invention is not limited to the delivery system and stents discussed below by way of example.

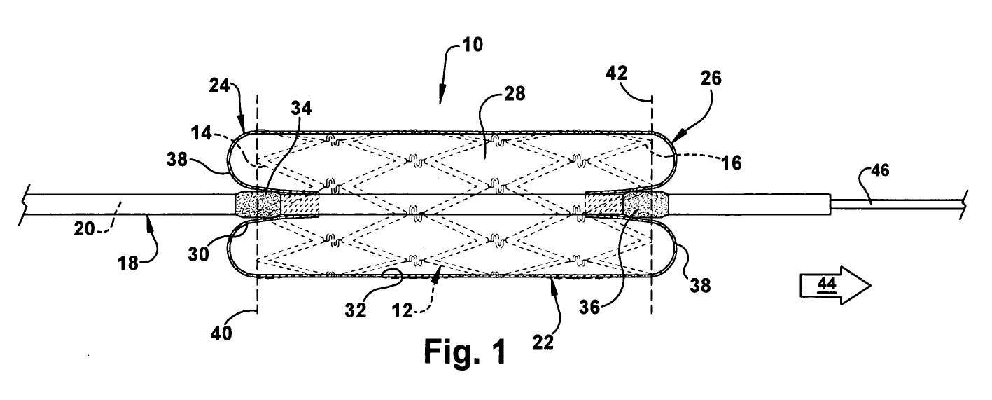

As shown in cross-section in FIG. 1, the apparatus 10 comprises a catheter shaft 18 having a lumen 20 and an inflatable balloon 22. The balloon 22 may be made of a conventional material, such as polytetrafluoroethylene, elastomeric materials including latex, silicone, polyolefin copolymers, or any other suitable balloon materials known in the art. Examples of balloon materials are disclosed in U.S. Pat. Nos. 5,830,182 and 5,500,181.

The balloon 22 has oppositely disposed first and second end portions 24 and 26 separated by an internal chamber 28. As is known in the art, the internal chamber 28 of the balloon 22 is in fluid communication with the lumen 20 in the catheter shaft 18 and receives inflation fluid to inflate the balloon. The balloon 22 has cylindrical inner and outer surfaces 30 and 32 that extend at least a portion of the distance between the first and second end portions 24 and 26. The cylindrical inner surface 30 of the balloon 22 is secured to the catheter shaft 18 at first and second attachment sites 34 and 36 adjacent the first and second end portions 24 and 26, respectively. The catheter shaft 18 may act cooperatively with the cylindrical inner and outer surfaces 30 and 32 to contain the inflation fluid. The balloon 22 is secured and sealed to the catheter shaft 18 at the first and second attachment sites 34 and 36 by suitable means, such as via heat bonding or an appropriate adhesive.

The first end portion 24 of the balloon 22 has a toroidal shape defined by an arcuate end surface 38 that intersects the cylindrical inner and outer surfaces 30 and 32 at a common radial plane 40. The attachment site 34 is located adjacent the first end portion 24 and the common radial plane 40. The balloon 22 is positioned within the stent 12, shown in phantom in FIGS. 1-3, so that the terminal edge 14 of the stent lies in the common radial plane 40.

The second end portion 26 of the balloon 22 has a toroidal shape defined by an arcuate end surface 38 that intersects the cylindrical inner and outer surfaces 30 and 32 at a second common radial plane 42. The second attachment site 36 is located adjacent the second end portion 26 and the second common radial plane 42. The balloon 22 is positioned within the stent 12 so that the second terminal edge 16 of the stent lies in the second radial plane 42.

To use the apparatus 10 in a vascular location, the expandable stent 12 is first mounted on the balloon 22 when the balloon is in a deflated configuration and the stent is in an unexpanded configuration (not shown). As discussed above, the balloon 22 is positioned within the stent 12 so that the terminal edges 14 and 16 of the stent will lie in the common radial planes 40 and 42, respectively, when the balloon 22 is inflated. The apparatus 10 may then be guided in a first direction 44 to the desired location in a known manner via a guidewire 46. Upon placement of the apparatus 10 at the desired location, the balloon 22 can then be expanded by a source of inflation fluid (not shown), pressure from which also expands the stent 12 to the expanded configuration shown in cross-section in FIG. 1. In this expanded configuration, the geometry of the balloon 22 and the placement of the stent 12 thereon ensure that, when the balloon is expanded, the terminal edges 14 and 16 of the stent 12 receive the full and uniform expansion pressure of the balloon. By ensuring full and uniform expansion pressure using the balloon 22 geometry of the present invention, the end portions 24 and 26 of the balloon 22 do not overhang/mushroom beyond the terminal edges 14 and 16, respectively, of the stent 12. This relationship, in turn, prevents damage to the vascular environment surrounding the apparatus 10 and reduces or eliminates edge restenosis.

Once the stent 12 is placed, the balloon 22 is at least partially deflated by removal of the inflation fluid. The stent 12 remains in place at the vascular location, and the balloon 22 can be withdrawn from the vascular location in a known manner.

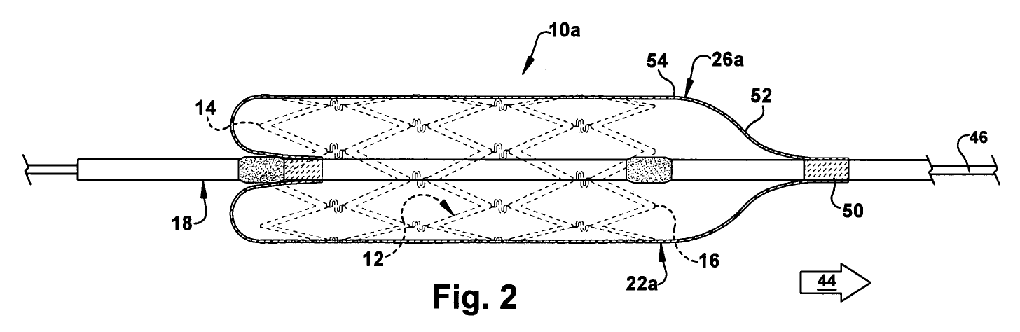

FIG. 2 is a cross-sectional side view of an alternative embodiment of the apparatus 10a. In FIG. 2, structures that are identical to structures in FIG. 1 use the same reference numbers, whereas structures that are similar but not identical carry the suffix “a”. In this embodiment of the apparatus 10a, the second end portion 26a of the balloon 22a has a funnel or conical shape. At the second end portion 26a, the balloon 22a is attached to the catheter shaft 18 at an attachment site 50 spaced apart from the expandable stent 12 along the catheter shaft 18. The balloon 22a has a tapered edge 52 with a variable diameter that leads to a balloon shoulder 54. The balloon shoulder 54 is the length of the balloon 22a that extends beyond the second terminal edge 16 of the stent 12. The tapered edge 52 of the conical second end portion 26a assists in smoothly moving the apparatus 10a through the bodily vessel when the apparatus 10a is guided in the first direction 44.

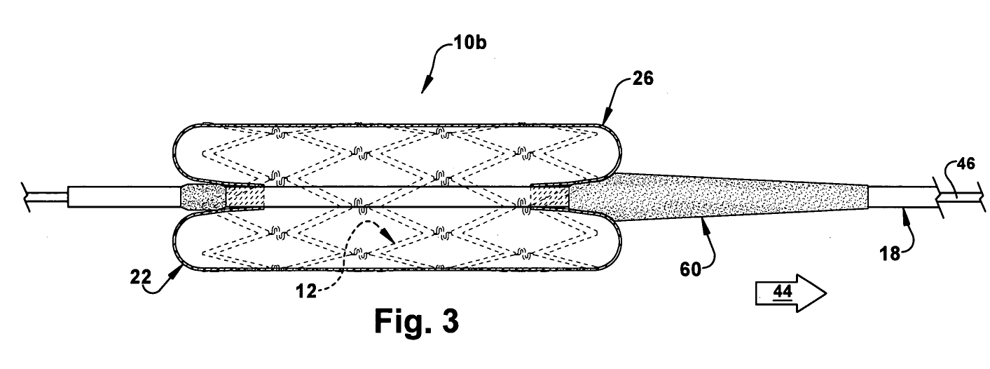

Now referring to FIG. 3, a cross-sectional view of another alternative embodiment of the apparatus 10b is shown. In FIG. 3, structures that are identical to structures in FIG. 1 use the same reference numbers, whereas structures that are similar but not identical carry the suffix “b”. In this embodiment, a nosepiece 60 mates with the second end portion 26 of the balloon 22. The nosepiece 60 may have a conical shape and should be positioned on the catheter shaft 18 in a mating relationship with the second end portion 26 of the apparatus 10b. In this position, the nosepiece 60 can prevent loose material within the bodily vessel from accumulating at the second end portion 26 when the apparatus 10b is guided in the first direction 44, and ease the passage of the apparatus 10b through the bodily vessel.

From the above description of the invention, those skilled in the art will perceive improvements, changes and modifications. For example, the apparatus 10 may be used in any suitable bodily vessel, including, but not limited to, coronary, mesentery, peripheral, or cerebral vasculature, veins, the gastrointestinal tract, the biliary tract, the urethra, the trachea, hepatic shunts and fallopian tubes. Such improvements, changes and modifications within the skill of the art are intended to be covered by the appended claims.

Having described the invention, we claim:

1. An apparatus for use with an expandable stent having oppositely disposed first and second terminal edges, said apparatus comprising:
a catheter shaft having a lumen; and
an inflatable balloon having oppositely disposed first and second end portions and an internal chamber, said internal chamber in said balloon being in fluid communication with said lumen in said catheter shaft for receiving inflation fluid to inflate said balloon, said balloon including cylindrical inner and outer surfaces that extend between said end portions, said cylindrical inner surface of said balloon being secured to said catheter shaft at first and second attachment sites adjacent said first and second end portions, respectively;
at least one of said first and second end portions of said balloon having a toroidal shape defined by an arcuate end surface that intersects said cylindrical inner and outer surfaces at a common radial plane;
a respective one of said attachment sites located adjacent said one end portion lying adjacent said common radial plane, said balloon being positionable within the stent so that one of the terminal edges of the stent lies in said common radial plane to ensure that, when said balloon is expanded, the one terminal edge receives full and uniform expansion pressure.

2. The apparatus of claim 1, wherein said second end portion of said balloon has a toroidal shape defined by an arcuate end surface that intersects said cylindrical inner and outer surfaces at a second common radial plane.

3. The apparatus of claim 1, wherein a respective one of said attachment sites is located adjacent said second end portion lying adjacent said second common radial plane, said balloon being positionable within the stent so that the other of the terminal edges of the stent lies in said second radial plane to ensure that, when said balloon is expanded, the other terminal edge receives full and uniform expansion pressure.

4. An apparatus for use in a bodily vessel, said apparatus comprising:
a catheter shaft having a lumen;
an inflatable balloon having oppositely disposed first and second end portions and an internal chamber, said internal chamber in said balloon being in fluid communication with said lumen in said catheter shaft for receiving inflation fluid to inflate said balloon, said balloon including cylindrical inner and outer surfaces that extend between said end portions, said cylindrical inner surface of said balloon being secured to said catheter shaft at first and second attachment sites adjacent said first and second end portions, respectively;
at least one of said first and second end portions of said balloon having a toroidal shape defined by an arcuate end surface that intersects said cylindrical inner and outer surfaces at a common radial plane, wherein a respective one of said attachment sites located adjacent said one end portion lying adjacent said common radial plane; and
an expandable stent having oppositely disposed first and second terminal edges, said stent being disposed about said balloon so that one of said terminal edges lies in said common radial plane to ensure that, when said balloon is expanded, said one terminal edge receives full and uniform expansion pressure to firmly engage the wall of the blood vessel.

5. The apparatus of claim 4, wherein said second end portion of said balloon has a toroidal shape defined by an arcuate end surface that intersects said cylindrical inner and outer surfaces at a second common radial plane.

6. The apparatus of claim 4, wherein a respective one of said attachment sites is located adjacent said second end portion lying adjacent said second common radial plane, said balloon being positionable within the stent so that the other of the terminal edges of the stent lies in said second radial plane to ensure that, when said balloon is expanded, the other terminal edge receives full and uniform expansion pressure.
